(19) United States
(12) Patent Application Publication
(10) Pub. No.: US 2015/0132515 A1
(43) Pub. Date: May 14, 2015

(54) POLYPROPYLENE-BASED FILM WITH IMPROVED MECHANICAL AND SEALING PROPERTIES AND METHOD OF MAKING SAME

(71) Applicant: Dow Global Technologies LLC, Midland, MI (US)

(72) Inventors: Claudia Hernandez, Lake Jackson, TX (US); Andy C. Chang, Houston, TX (US); Teresa Bernal Lara, Lincolnshire, IL (US)

(21) Appl. No.: 14/394,302

(22) PCT Filed: Mar. 14, 2013
(86) PCT No.: PCT/US2013/031177

§ 371 Date: Oct. 14, 2014

Related U.S. Application Data
(60) Provisional application No. 61/645,373, filed on May 10, 2012.

(51) Int. Cl. B32B 27/08; B29C 47/06; B32B 27/32

(52) U.S. Cl. CPC B32B 27/08; B32B 27/32; B29C 47/06; B32B 2250/03; B32B 2250/04; B32B 2250/05; B32B 2250/40; B32B 2307/558; B32B 2307/5825; B32B 2323/10; B32B 2307/31; B29K 2023/12
USPC 428/35.7; 428/516; 428/221; 264/173.19; 156/73.1

(57) ABSTRACT

A polypropylene-based film comprising at least three layers characterized by two skin layers and at least one core layer, wherein the at least one core layer and/or at least one skin layer comprises a polymer blend of: (i) a first propylene-based polymer which comprises (A) at least 60 weight percent (wt %) units derived from propylene, and (B) from 5 to 40 wt % units derived from ethylene, and wherein the first propylene-based polymer is characterized by a melting temperature (Tm) of less than or equal to 110° C., and (ii) a second propylene-based polymer selected from the group consisting of rPP and hPP and characterized by certain mechanical and/or sealing properties is provided. Also provided is a method of making the polypropylene-based film.

### FIELD OF INVENTION

The instant invention relates to a polypropylene-based film, and method of making such film.

### BACKGROUND OF THE INVENTION

Conventional multi-layer films using random polypropylene (rPP) and/or propylene homopolymer (hPP) in the skin, i.e. outermost, layer, are known. Such conventional films are prone to failure due to the propensity of rPP and hPP to tear during mechanical stretching.

Films with improved toughness, tear resistance, and puncture resistance would benefit converters by reduced product failure, off grade, and disruptions to production (down time). Brand owners would also benefit from improved films based on reduced disruptions during article fabrication with films and laminates, as well as improved sealing properties. Finally, consumers would benefit from such improved films based on reduced article failure in end-use.

### SUMMARY OF THE INVENTION

The instant invention is a polypropylene-based film, and method of making such film.

In one embodiment, the instant invention provides a polypropylene-based film comprising: at least three layers characterized by two skin layers and at least one core layer, wherein the at least one core layer comprises a blend of: (i) a first propylene-based polymer which comprises (A) at least 60 weight percent (wt %) units derived from propylene, and (B) from 5 to 40 wt % units derived from ethylene, and wherein the first propylene-based polymer is characterized by a melting temperature (Tm) of less than or equal to 110° C., and (ii) a second propylene-based polymer selected from the group consisting of rPP and hPP, and wherein the film exhibits at least one of the following characteristics: (a) change in Elmendorf tear machine direction (Elmendorf MD) of equal to or greater than 100% over a three-layer film comprising solely polypropylene consisting essentially of rPP, hPP or combinations thereof; (b) change in dart impact resistance of equal to or greater than 60% over a three-layer film comprising solely polypropylene consisting essentially of rPP, hPP or combinations thereof; (c) change in 2% secant modulus of equal to or greater than 15% over a three-layer film comprising solely polypropylene consisting essentially of rPP, hPP or combinations thereof; (d) change in puncture resistance of equal to or greater than 40% over a three-layer film comprising solely polypropylene consisting essentially of rPP, hPP or combinations thereof; (e) normalized Elmendorf MD of equal to or greater than 25, g/mils; (f) dart impact resistance of equal to or greater than 400 g; (g) 2% Secant modulus (MD) at least 20000 psi; and (h) puncture equal to or greater than 140 ft-lb/in³.

### BRIEF DESCRIPTION OF THE DRAWINGS

For the purpose of illustrating the invention, there is shown in the drawings a form that is exemplary; it being understood, however, that this invention is not limited to the precise arrangements and instrumentalities shown.

FIG. 1 is graph illustrating the effect of increasing % weight ethylene content of VERSIFY in core layer on the normalized Elmendorf MD and CD tear strength, dart impact resistance, puncture and MD 2% secant modulus for each of Inventive Examples 1-3;

FIG. 2 is a graph illustrating the effect of increasing the % weight of DE 3300 in skin layer on the normalized Elmendorf MD and CD tear strength, dart impact resistance, puncture and MD 2% secant modulus for each of Inventive Examples 4-7;

FIG. 3 is a graph illustrating the effect of increasing VERSIFY 3300 content in the skin layers on the heat seal property of Inventive Examples 4-7;

FIG. 4 is a graph illustrating the effect of increasing VERSIFY 3300 content in the skin layers on the hot tack strength of Inventive Examples 4-7;

FIGS. 5a and 5b are graphs illustrating the effect of aging on Elmendorf tear in machine direction (FIG. 5a) and cross-direction (FIG. 5b) for three layer films (A/B/A) which differ from each other only in the type of VERSIFY used in the core layer;

FIGS. 6a and 6b are graphs illustrating the effect of aging on Elmendorf tear in machine direction (FIG. 6a) and cross-direction (FIG. 6b)s for three layer films which differ from each other in the percentage of VERSIFY DE 3300 in the skin layers;

FIG. 7a is a graph illustrating the effect of adding VERSIFY up to 50% in hPP or rPP on normalized Elmendorf tear in machine direction;

FIG. 7b is a graph illustrating the effect of adding VERSIFY into hPP or rPP on the Elmendorf tear in the cross-direction;

FIG. 8 is a graph illustrating the effect of adding VERSIFY on the puncture resistance of the polypropylene cast films;

FIG. 9 is a graph illustrating the effect of increasing VERSIFY content in hPP or rPP on dart resistance;

FIG. 10 is a graph illustrating the effect of the addition of VERSIFY into hPP on 2% secant modulus in machine direction;

FIG. 11 are graphs illustrating the optical properties of PP/VERSIFY™ monolayer cast films; and

FIGS. 12 and 13 show the effect of aging on Elmendorf tear of VERSIFY/hPP blends and VERSIFY/rPP blends, respectively.

### DETAILED DESCRIPTION OF THE INVENTION

As used herein, the term “heat seal initiation temperature” means the temperature at which 2 lb/in (8.8 N/2.54 mm) heat seal strength is achieved.

The instant invention is a polypropylene-based film and method of making the same.

In a first embodiment, the instant invention provides a polypropylene-based film which comprises: at least three layers characterized by two skin layers and at least one core layer, wherein the at least one core layer comprises a polymer blend of: (i) a first propylene-based polymer which comprises (A) at least 60 weight percent (wt %) units derived from propylene, and (B) from 5 to 40 wt % units derived from ethylene, and wherein the first propylene-based polymer is characterized by a melting temperature (Tm) of less than or equal to 110° C., and (ii) a second propylene-based polymer selected from the group consisting of rPP, hPP, and combinations thereof, and wherein the film exhibits at least one of the following characteristics: (a) change in Elmendorf tear MD of equal to or greater than 100% over a three-layer film comprising solely polypropylene consisting essentially of rPP, hPP or combinations thereof; (b) change in dart impact resistance of equal to or greater than 60% over a three-layer film comprising solely polypropylene consisting essentially of rPP, hPP or combinations thereof; (c) change in 2% secant modulus of equal to or greater than 15% over a three-layer film comprising solely polypropylene consisting essentially of rPP, hPP or combinations thereof; (d) change in puncture resistance of equal to or greater than 40% over a three-layer film comprising solely polypropylene consisting essentially of rPP, hPP or combinations thereof; (e) normalized Elmendorf MD of equal to or greater than 25 g/mils; (f) dart impact resistance of equal to or greater than 400 g; (g) 2% Secant modulus (MD) at least 20000 psi; and (h) puncture equal to or greater than 140 ft-lb/in³.

An alternative embodiment of the instant invention further provides a polypropylene-based film comprising: at least three layers characterized by two skin layers and at least one core layer, wherein at least one of the two skin layers comprises a polymer blend of: (i) a first propylene-based polymer which comprises (A) at least 60 weight percent (wt %) units derived from propylene, and (B) from 5 to 40 wt % units derived from ethylene, and wherein the first propylene-based polymer is characterized by a Tm of less than or equal to 110° C.; and (ii) a second propylene-based polymer selected from the group consisting of rPP, hPP, and combinations thereof; and wherein the film exhibits at least one of the following characteristics: (a) change in Elmendorf tear MD of equal to or greater than 100% over a three-layer film comprising solely polypropylene consisting essentially of rPP, hPP or combinations thereof; (b) change in dart impact resistance of equal to or greater than 60% over a three-layer film comprising solely polypropylene consisting essentially of rPP, hPP or combinations thereof; (c) change in 2% secant modulus of equal to or greater than 15% over a three-layer film comprising solely polypropylene consisting essentially of rPP, hPP or combinations thereof; (d) change in puncture resistance of equal to or greater than 40% over a three-layer film comprising solely polypropylene consisting essentially of rPP, hPP or combinations thereof; (e) reduction in heat seal initiation temperature of equal to or greater than 10° C. in comparison to a three-layer film comprising solely polypropylene consisting essentially of rPP, hPP or combinations thereof; (f) reduction in hot tack initiation temperature of equal to or greater than 20° C. in comparison a three-layer film comprising solely polypropylene consisting essentially of rPP, hPP or combinations thereof; (g) normalized Elmendorf MD of equal to or greater than 25, g/mils; (h) dart impact resistance of equal to or greater than 400 g; (i) 2% Secant modulus (MD) at least 20000 psi; and (j) puncture equal to or greater than 140 ft-lb/in³; (k) heat seal initiation temperature of less than 130° C.; (l) hot tack initiation temperature of less than 130° C.; and (m) hot tack strength greater than 2 N/in.

All individual values and subranges at least 60 percent by weight (“weight percent” or “wt %”) units derived from propylene in the first propylene-based polymer of the at least one core layer are included herein and disclosed herein; for example, the amount of units derived from propylene in the first propylene-based polymer of the at least one core layer can be from a lower limit of 60, 65, 70, 75, 80, 85, 90 or 95 wt %. For example, the amount of units derived from propylene in the first propylene-based polymer of the at least one core layer can be at least 60 wt %, or in the alternative, the amount of units derived from propylene in the first propylene-based polymer of the at least one core layer may be at least 75 wt %, or in the alternative, the amount of units derived from propylene in the first propylene-based polymer of the at least one core layer may be at least 80 wt %.

All individual values and subranges from 5 to 40 wt % units derived from ethylene in the first propylene-based polymer of the at least one core layer are included herein and disclosed herein; for example, the amount of units derived from ethylene in the first propylene-based polymer of the at least one core layer can be from a lower limit of 5, 10, 15, 20, 25, 30 or 35 wt % to an upper limit of 10, 15, 20, 25, 30, 35 or 40 wt %. For example, the amount of units derived from ethylene in the first propylene-based polymer of the at least one core layer can be from 5 to 40 wt %, or in the alternative, the amount of units derived from ethylene in the first propylene-based polymer of the at least one core layer may be from 15 to 35 wt %, or in the alternative, the amount of units derived from ethylene in the first propylene-based polymer of the at least one core layer may be from 20 to 40 wt %.

All individual values and subranges of Tm from less than or equal to 110° C. of the first propylene-based polymer of the at least one core layer are included herein and disclosed herein; for example, the Tm of the first propylene-based polymer of the at least one core layer can be from an upper limit of 110, 109, 108, 107 or 106° C. For example, the Tm of the first propylene-based polymer of the at least one core layer may be less than or equal to 110° C., or in the alternative, the Tm of the first propylene-based polymer of the at least one core layer may be less than or equal to 108° C., or in the alternative, the Tm of the first propylene-based polymer of the at least one core layer may be less than or equal to 106° C.

In those embodiments in which the polypropylene-based film exhibits a change in Elmendorf tear MD of equal to or greater than 100% over a three-layer film comprising solely polypropylene consisting essentially of rPP, hPP or combinations thereof, all values and subranges of equal to or greater than 100% are included herein and disclosed herein. For example, the change in Elmendorf tear MD may be from a lower limit of 100%, 110%, 130%, 150%, 170%, or 190% over a three-layer film comprising solely polypropylene consisting essentially of rPP, hPP or combinations thereof.

In those embodiments in which the polypropylene-based film exhibits a change in dart impact resistance of equal to or greater than 60% over a three-layer film comprising solely polypropylene consisting essentially of rPP, hPP or combinations thereof, all values and subranges of equal to or greater than 60% are included herein and disclosed herein. For example, the change in dart impact resistance may be from a lower limit of 60%, 65%, 70%, 80%, 85%, or 90% over a three-layer film comprising solely polypropylene consisting essentially of rPP, hPP or combinations thereof.

In those embodiments in which the polypropylene-based film exhibits a change in 2% secant modulus of equal to or greater than 15% over a three-layer film comprising solely polypropylene consisting essentially of rPP, hPP or combinations thereof, all values and subranges of equal to or greater than 15% are included herein and disclosed herein. For example, the change in 2% secant modulus may be from a lower limit of 15%, 20%, 30%, 35%, or 50% over a three-layer film comprising solely polypropylene consisting essentially of rPP, hPP or combinations thereof.

In those embodiments in which the polypropylene-based film exhibits a change in puncture resistance of equal to or greater than 40% over a three-layer film comprising solely polypropylene consisting essentially of rPP, hPP or combinations thereof, all values and subranges of equal to or greater than 40% are included herein and disclosed herein. For example, the change in Elmendorf tear MD may be from a lower limit of 40%, 50%, 55%, 60%, 70%, or 75% over a three-layer film comprising solely polypropylene consisting essentially of rPP, hPP or combinations thereof.

In those embodiments in which the polypropylene-based film exhibits a normalized Elmendorf MD of equal to or greater than 25 g/mils, all values and subranges of equal to or greater than 25 g/mils are included herein and disclosed herein. For example, the polypropylene-based film may exhibit a normalized Elmendorf MD of equal to or greater than 50 g/mils, or in the alternative, the polypropylene-based film may exhibit a normalized Elmendorf MD of equal to or greater than 75 g/mils, or in the alternative, the polypropylene-based film may exhibit a normalized Elmendorf MD of equal to or greater than 100 g/mils.

In those embodiments in which the polypropylene-based film exhibits a dart impact resistance of equal to or greater than 400 g, all values and subranges of equal to or greater than 400 g are included herein and disclosed herein. For example, the polypropylene-based film may exhibit a dart impact of equal to or greater than 400 g, or in the alternative, the polypropylene-based film may exhibit a dart impact of equal to or greater than 450 g, or in the alternative, the polypropylene-based film may exhibit a dart impact of equal to or greater than 500 g, or in the alternative, the polypropylene-based film may exhibit a dart impact of equal to or greater than 550 g.

In those embodiments in which the polypropylene-based film exhibits a 2% Secant modulus (MD) at least 20000 psi, all values of equal to and greater than 20000 psi are included herein and disclosed herein. For example, the polypropylene-based film may exhibit a 2% Secant modulus (MD)of equal to or greater than 20000 psi, or in the alternative, the polypropylene-based film may exhibit a 2% Secant modulus (MD) of equal to or greater than 25000 psi, or in the alternative, the polypropylene-based film may exhibit a 2% Secant modulus (MD) of equal to or greater than 30000 psi, or in the alternative, the polypropylene-based film may exhibit a 2% Secant modulus (MD) of equal to or greater than 35000 psi.

In those embodiments in which the polypropylene-based film exhibits a puncture equal to or greater than 140 ft-lb/in³, all values of equal to or greater than 140 ft-lb/in³ are included herein and disclosed herein. For example, the polypropylene-based film may exhibit a puncture of equal to or greater than 140 ft-lb/in³, or in the alternative, the polypropylene-based film may exhibit a puncture of equal to or greater than 160 ft-lb/in³, or in the alternative, the polypropylene-based film may exhibit a puncture of equal to or greater than 180 ft-lb/in³, or in the alternative, the polypropylene-based film may exhibit a puncture of equal to or greater than 200 ft-lb/in³.

In those embodiments in which the polypropylene-based film exhibits a reduction in heat seal initiation temperature of equal to or greater than 10° C. in comparison a three-layer film comprising solely polypropylene consisting essentially of rPP, hPP or combinations thereof, all values and subranges of equal to or greater than 10° C. are included herein and disclosed herein. For example, reduction in heat seal initiation temperature may be from a lower limit of 10, 14, 18, or 20° C. in comparison a three-layer film comprising solely polypropylene consisting essentially of rPP, hPP or combinations thereof

In those embodiments in which the polypropylene-based film exhibits a reduction in hot tack initiation temperature of equal to or greater than 20° C. in comparison a three-layer film comprising solely polypropylene consisting essentially of rPP, hPP or combinations thereof, all values and subranges of equal to or greater than 20° C. are included herein and disclosed herein. For example, reduction in hot tack initiation temperature may be from a lower limit of 20, 24, 28, or 30° C. in comparison a three-layer film comprising solely polypropylene consisting essentially of rPP, hPP or combinations thereof.

In those embodiments in which the polypropylene-based film exhibits a heat seal initiation temperature of less than 130° C., all values and subranges of less than 130° C. are included herein and disclosed herein. For example, the polypropylene-based film may exhibit a heat seal initiation temperature of less than 130° C., or in the alternative, the polypropylene-based film may exhibit a heat seal initiation temperature of less than 120° C., or in the alternative, the polypropylene-based film may exhibit a heat seal initiation temperature of less than 115° C., or in the alternative, the polypropylene-based film may exhibit a heat seal initiation temperature of less than 112° C.

In those embodiments in which the polypropylene-based film exhibits a hot tack initiation temperature of less than 130° C., all values and subranges of less than 130° C. are included herein and disclosed herein. For example, the polypropylene-based film may exhibit a hot tack initiation temperature of less than 130° C., or in the alternative, the polypropylene-based film may exhibit a hot tack initiation temperature of less than 120° C., or in the alternative, the polypropylene-based film may exhibit a hot tack initiation temperature of less than 115° C., or in the alternative, the polypropylene-based film may exhibit a hot tack initiation temperature of less than 112° C.

In those embodiments in which the polypropylene-based film exhibits a hot tack strength greater than 2 N/in, all values and subranges of greater than 2N/in are included herein and disclosed herein. For example, the polypropylene-based film may exhibit a hot tack strength greater than 2 N/in, or in the alternative, the polypropylene-based film may exhibit a hot tack strength greater than 3 N/in, or in the alternative, the polypropylene-based film may exhibit a hot tack strength greater than 4 N/in, or in the alternative, the polypropylene-based film may exhibit a hot tack strength greater than 5 N/in.

In an alternative embodiment, the instant invention further provides a method for making a polypropylene-based film comprising: selecting a first propylene-based polymer; selecting a second propylene-based polymer; blending the first and second propylene-based polymers to form a polymer blend; forming the polypropylene-based film wherein at least one layer comprises the blend, wherein the first propylene-based polymer comprises from 5 to 40 wt % units derived from ethylene and the second propylene-based polymer is selected from the group consisting of rPP, hPP and combinations thereof.

Exemplary polymer blends useful in various embodiments of the invention are commercially available under the names VERSIFY (available from The Dow Chemical Company), VISTAMAXX (available from ExxonMobil Chemical Co.), TAFMER XM and NOTIO (available from Mitsui Chemical Company), and LMPP (available from Idemitsu Kosan Company).

All individual values and subranges from 5 to 40 wt % units derived from ethylene in the first propylene-based polymer are included herein and disclosed herein; for example, the amount of units derived from ethylene in the first propylene-based polymer can be from a lower limit of 5, 10, 15, 20, 25, 30, or 35 wt % to an upper limit of 10, 15, 20, 25, 30, 35, or 40 wt %. For example, the amount of units derived from ethylene in the first propylene-based polymer may be in the range of from 5 to 40 wt %, or in the alternative, the amount of units derived from ethylene in the first propylene-based polymer may be in the range of from 5 to 25 wt %, or in the alternative, the amount of units derived from ethylene in the first propylene-based polymer may be in the range of from 20 to 40 wt %.

In an alternative embodiment, the instant invention provides a polypropylene-based film and a method of making the same, in accordance with any of the preceding embodiments, except that the film is an extrusion coating and the first polypropylene-based polymer has an MFR of from 8 to 40 g/10 min.

All individual values and subranges from 8 to 40 g/10 min MFR of the first propylene-based polymer used in an extrusion coating film are included herein and disclosed herein; for example, the MFR of the first propylene-based polymer used in an extrusion coating film can be from a lower limit of 8, 10, 14, 18, 24, 28, 32, or 36 g/10min to an upper limit of 9, 13, 19, 29, 33 or 40 g/10 min. For example, the MFR of the first propylene-based polymer used in an extrusion coating film may be in the range of from 8 to 40 g/10 min, or in the alternative, the MFR of the first propylene-based polymer used in an extrusion coating film may be in the range of from 15 to 35 g/10 min, or in the alternative, the MFR of the first propylene-based polymer used in an extrusion coating film may be in the range of from 8 to 25 g/10 min.

In an alternative embodiment, the instant invention provides a polypropylene-based film and a method of making the same, in accordance with any of the preceding embodiments, except that the film is a cast film and the first polypropylene-based polymer has an MFR from 3 to 40 g/10 min.

All individual values and subranges from 3 to 40 g/10 min MFR of the first propylene-based polymer used in a cast film are included herein and disclosed herein; for example, the MFR of the first propylene-based polymer used in a cast film can be from a lower limit of 3, 5, 8, 10, 14, 18, 24, 28, 32, or 36 g/10 min to an upper limit of 9, 13, 19, 29, 33 or 40 g/10 min. For example, the MFR of the first propylene-based polymer used in a cast film may be in the range of from 3 to 40 g/10 min, or in the alternative, the MFR of the first propylene-based polymer used in a cast film may be in the range of from 5 to 35 g/10 min, or in the alternative, the MFR of the first propylene-based polymer used in a cast film may be in the range of from 20 to 40 g/10 min.

In an alternative embodiment, the instant invention provides a polypropylene-based film and a method of making the same, in accordance with any of the preceding embodiments, except that the film is a blown film and the first polypropylene-based polymer has an MFR of from 0.3 to 10 g/10 min.

All individual values and subranges from 0.3 to 10 g/10 min MFR of the first propylene-based polymer used in a blown film are included herein and disclosed herein; for example, the MFR of the first propylene-based polymer used in a blown film can be from a lower limit of 0.3, 1, 2. 3.2, 4.4, 5, 6, 7.5, 8, 7, or 9 g/10 min to an upper limit of 1, 3, 5, 7, or 10 g/10 min. For example, the MFR of the first propylene-based polymer used in a blown film may be in the range of from 0.3 to 10 g/10 min, or in the alternative, the MFR of the first propylene-based polymer used in a blown film may be in the range of from 1.5 to 7 g/10 min, or in the alternative, the MFR of the first propylene-based polymer used in a blown film may be in the range of from 5 to 9 g/10 min.

In an alternative embodiment, the instant invention provides a polypropylene-based film and a method of making the same, in accordance with any of the preceding embodiments, except that the film comprises three layers characterized by two skin layers and one core layer.

In an alternative embodiment, the instant invention provides a polypropylene-based film and a method of making the same, in accordance with any of the preceding embodiments, except that the film comprises four layers characterized by two skin layers and two core layers.

In an alternative embodiment, the instant invention provides a polypropylene-based film and a method of making the same, in accordance with any of the preceding embodiments, except that the film comprises five, seven or nine layers characterized by two skin layers and three, five or seven core layers respectively. In a further embodiment, the instant invention provides for multilayer films comprising two skin layers and at least one core layer.

In an alternative embodiment, the instant invention provides a polypropylene-based film and a method of making the same, in accordance with any of the preceding embodiments, except that the film further comprises at least one tie layer disposed between at least one skin layer and at least one core layer. As used herein, the term “tie layer” refers to an intermediate layer of a multilayer film wherein the intermediate layer can promote the adhesion between two adjacent layers of the intermediate layer.

The tie layer can be any layer that can promote the adhesion between its two adjacent layers. Some non-limiting examples of suitable polymers for the tie layer include ethylene/vinyl acetate copolymers, ethylene/methyl acrylate copolymers, ethylene/butyl acrylate copolymers, and polar group functionalized polymers. In some embodiments, the thickness of the tie layer is from about 1 to 20% of the total film thickness. All values and subranges from about 1 to 20% of the total film thickness are included herein and disclosed herein.

In an alternative embodiment, the instant invention provides a polypropylene-based film and a method of making the same, in accordance with any of the preceding embodiments, except that the total skin thickness comprises from 4 to 40 percent (%) of the total thickness of the film.

All individual values and subranges 4 to 40% are included herein and disclosed herein; for example, the total skin thickness can be from a lower limit of 4, 10, 15, 20, 25, 30, or 35% of the total thickness of the film to an upper limit of 5, 11, 16, 21, 26, 31, 36 or 40% of the total thickness of the film. For example, the total skin thickness may be in the range of from 4 to 40% of the total film thickness, or in the alternative, the total skin thickness may be in the range of from 14 to 26% of the total film thickness, or in the alternative, the total skin thickness may be in the range of from 4 to 16% of the total film thickness.

In an alternative embodiment, the instant invention provides a polypropylene-based film and a method of making the same, in accordance with any of the preceding embodiments, except that the film is formed by a co-extrusion method selected from the group consisting of cast extrusion, blown film extrusion and extrusion coating.

In an alternative embodiment, the instant invention provides a polypropylene-based film and a method of making the same, in accordance with any of the preceding embodiments, except that the film is formed by a lamination method selected from the group consisting of thermal, ultrasonic and adhesive lamination.

The polymer blends used in embodiments of the invention may be made by any appropriate method, such as dry blending, compounding, or melt kneading, melt blending, and solvent blending.

Embodiments of the inventive films may be made by conventional fabrication techniques, e.g. simple bubble extrusion, biaxial orientation processes (such as tenter frames or double bubble processes), simple cast/sheet extrusion, coextrusion, and lamination. Conventional simple bubble extrusion processes (also known as hot blown film processes) are described, for example, in The Encyclopedia of Chemical Technology, Kirk-Othmer, Third Edition, John Wiley & Sons, New York, 1981, Vol. 16, pp. 416-417 and Vol. 18, pp. 191-192, the disclosures of which are incorporated herein by reference. Biaxial orientation film manufacturing processes such as described in the “double bubble” process of U.S. Pat. No. 3,456,044 (Pahlke), and the processes described in U.S. Pat. No. 4,352,849 (Mueller), U.S. Pat. Nos. 4,820,557 and 4,837,084 (both to Warren), U.S. Pat. No. 4,865,902 (Golike et al.), U.S. Pat. No. 4,927,708 (Herran et al.), U.S. Pat. No. 4,952,451 (Mueller), and U.S. Pat. Nos. 4,963,419 and 5,059,481 (both to Lustig et al.), the disclosures of which are incorporated herein by reference, can also be used to make the novel film structures of this invention. Biaxially oriented film structures can also be made by a tenter-frame technique, such as that used for oriented polypropylene.

Other multilayer film manufacturing techniques for food packaging applications are described in Packaging Foods With Plastics by Wilmer A. Jenkins and James P. Harrington (1991), pp. 19-27, and in “Coextrusion Basics” by Thomas I. Butler, Film Extrusion Manual Process, Materials, Properties. pp. 31-80 (published by TAPPI Press (1992)) the disclosures of which are incorporated herein by reference.

A multilayer film may be obtained from three or more monolayer films, each of which is separately produced via, for example, a tubular film (i.e., blown film techniques) or flat die (i.e. cast film) as described by K. R. Osborn and W. A. Jenkins in “Plastic Films, Technology and Packaging Applications” (Technomic Publishing Co., Inc. (1992)), the disclosures of which are incorporated herein by reference, wherein the separate films are formed into a multi-layer film, by way of, for example, post-extrusion lamination

Extrusion coating is yet another technique for producing films. Similar to cast film, extrusion coating is a flat die technique. The inventive films can be extrusion coated onto a substrate either in the form of a coextruded extrudate according to, for example, the processes described in U.S. Pat. No. 4,339,507 incorporated herein by reference. Utilizing multiple extruders or by passing the various substrates through the extrusion coating system several times can result in multiple polymer layers. Lamination techniques may also be used in addition to or in combination with a coextrusion step.

In an alternative embodiment, the instant invention provides a polypropylene-based film and a method of making the same, in accordance with any of the preceding embodiments, except that one or more layers of the inventive film further comprises one or more components selected from the group consisting of slip agents, pigments, fibers, carbon black, mineral oil extenders, antistatic agents, dyes, plasticizers, oils, waxes, antioxidants, UV stabilizers, colorants, fillers, flow aids, coupling agents, crosslinking agents, surfactants, solvents, anti-blocking agents, lubricants, antifogging agents, nucleating agents, flame retardants, and combinations thereof.

In an alternative embodiment, the instant invention provides a polypropylene-based film and a method of making the same, in accordance with any of the preceding embodiments, except that the films are cross-linked, before or after orientation, by any means known in the art, including, but not limited to, electron-beam irradiation, beta irradiation, gamma irradiation, corona irradiation, silanes, peroxides, allyl compounds and UV radiation with or without crosslinking catalyst. U.S. Pat. Nos. 6,803,014 and 6,667,351 disclose electron-beam irradiation methods that can be used in embodiments of the invention.

In an alternative embodiment, the instant invention provides a polypropylene-based film and a method of making the same, in accordance with any of the preceding embodiments, except that one or more layers of the film or the film as a whole may be subjected to treatments to orient the film, e.g., uniaxially or biaxially.

In an alternative embodiment, the instant invention provides a polyolefin-based elastic film and a method of making the same, in accordance with any of the preceding embodiments, except that the polyolefin-based film further comprises at least one core layer which comprises one or more components selected from the group consisting of styrene-isoprene-styrene polymers, styrene-ethylene-butadiene-styrene polymers, styrene-butadiene-styrene polymers, styrene-ethylenepropylene-styrene, styrene-ethylenepropylene-styrene-ethylenepropylene, hydrogenated polybutadiene polymers and hydrogenated poly-isoprene/butadiene polymers.

Some typical end use applications for the inventive films include food and household packaging, coated fabrics, hygiene films (i.e. diaper backsheets, feminine napkin backsheets, adult incontinence backsheet films, individual article packaging film), layers in elastic films, and laminates with elastic films and fibers.

In the case of hygiene backsheet films, the function is to act as a liquid impermeable layer as described in U.S. Patent Application Publication No. 20050192549 for the purpose of preventing “fluids absorbed into the device from wetting through to the user's garments.” The films of the instant invention when fabricated into a liquid impermeable film have the advantage of higher modulus compared to conventional polyethylene film. This enables the substitution of a thinner film (also known as downgauging or reduction of basis weight) while still fulfilling the same function. Compared to polypropylene backsheets as described by Keith Brechtelsbauer of Berry Plastics in his paper “Innovation Trends in Film-Based Outer Cover Designs for Absorbent Hygiene Products” at INSIGHT 2010 (Charlotte N.C.), edge damage is less recoverable because of less stretch and tear resistance. While not intended to be limited by theory, the films of the present invention with combination of tear resistance, puncture resistance, and dart impact resistance, and modulus mitigates problems identified by Brechtelsbauer.

In an alternative embodiment, the instant invention provides a polypropylene-based film consisting essentially of: three layers characterized by two skin layers and at least one core layer, wherein the at least one core layer comprises a polymer blend of: (i) a first propylene-based polymer which comprises (A) at least 60 weight percent (wt %) units derived from propylene, and (B) from 5 to 40 wt % units derived from ethylene, and wherein the first propylene-based polymer is characterized by a melting temperature (Tm) of less than or equal to 110° C., and (ii) a second propylene-based polymer selected from the group consisting of rPP, hPP, and combinations thereof, and wherein the film exhibits at least one of the following characteristics: (a) change in Elmendorf tear MD of equal to or greater than 100% over a three-layer film comprising solely polypropylene consisting essentially of rPP, hPP or combinations thereof; (b) change in dart impact resistance of equal to or greater than 60% over a three-layer film comprising solely polypropylene consisting essentially of rPP, hPP or combinations thereof; (c) change in 2% secant modulus of equal to or greater than 15% over a three-layer film comprising solely polypropylene consisting essentially of rPP, hPP or combinations thereof; (d) change in puncture resistance of equal to or greater than 40% over a three-layer film comprising solely polypropylene consisting essentially of rPP, hPP or combinations thereof; (e) normalized Elmendorf MD of equal to or greater than 25 g/mils; (f) dart impact resistance of equal to or greater than 400 g; (g) 2% Secant modulus (MD) at least 20000 psi; and (h) puncture equal to or greater than 140 ft-lb/in³.

In an alternative embodiment, the instant invention provides a polypropylene-based film comprising: at least three layers characterized by two skin layers and at least one core layer, wherein the at least one core layer consists essentially of a polymer blend of: (i) a first propylene-based polymer which comprises (A) at least 60 weight percent (wt %) units derived from propylene, and (B) from 5 to 40 wt % units derived from ethylene, and wherein the first propylene-based polymer is characterized by a melting temperature (Tm) of less than or equal to 110° C., and (ii) a second propylene-based polymer selected from the group consisting of rPP, hPP, and combinations thereof, and wherein the film exhibits at least one of the following characteristics: (a) change in Elmendorf tear MD of equal to or greater than 100% over a three-layer film comprising solely polypropylene consisting essentially of rPP, hPP or combinations thereof; (b) change in dart impact resistance of equal to or greater than 60% over a three-layer film comprising solely polypropylene consisting essentially of rPP, hPP or combinations thereof; (c) change in 2% secant modulus of equal to or greater than 15% over a three-layer film comprising solely polypropylene consisting essentially of rPP, hPP or combinations thereof; (d) change in puncture resistance of equal to or greater than 40% over a three-layer film comprising solely polypropylene consisting essentially of rPP, hPP or combinations thereof; (e) normalized Elmendorf MD of equal to or greater than 25 g/mils; (f) dart impact resistance of equal to or greater than 400 g; (g) 2% Secant modulus (MD) at least 20000 psi; and (h) puncture equal to or greater than 140 ft-lb/in³.

Another alternative embodiment of the instant invention further provides a polypropylene-based film consisting essentially of: three layers characterized by two skin layers and at least one core layer, wherein at least one of the two skin layers comprises a polymer blend of: (i) a first propylene-based polymer which comprises (A) at least 60 weight percent (wt %) units derived from propylene, and (B) from 5 to 40 wt % units derived from ethylene, and wherein the first propylene-based polymer is characterized by a Tm of less than or equal to 110° C.; and (ii) a second propylene-based polymer selected from the group consisting of rPP, hPP, and combinations thereof; and wherein the film exhibits at least one of the following characteristics: (a) change in Elmendorf tear MD of equal to or greater than 100% over a three-layer film comprising solely polypropylene consisting essentially of rPP, hPP or combinations thereof; (b) change in dart impact resistance of equal to or greater than 60% over a three-layer film comprising solely polypropylene consisting essentially of rPP, hPP or combinations thereof; (c) change in 2% secant modulus of equal to or greater than 15% over a three-layer film comprising solely polypropylene consisting essentially of rPP, hPP or combinations thereof; (d) change in puncture resistance of equal to or greater than 40% over a three-layer film comprising solely polypropylene consisting essentially of rPP, hPP or combinations thereof; (e) reduction in heat seal initiation temperature of equal to or greater than 10° C. in comparison to a three-layer film comprising solely polypropylene consisting essentially of rPP, hPP or combinations thereof; (f) reduction in hot tack initiation temperature of equal to or greater than 20° C. in comparison a three-layer film comprising solely polypropylene consisting essentially of rPP, hPP or combinations thereof; (g) normalized Elmendorf MD of equal to or greater than 25, g/mils; (h) dart impact resistance of equal to or greater than 400 g; (i) 2% Secant modulus (MD) at least 20000 psi; and (j) puncture equal to or greater than 140 ft-lb/in³; (k) heat seal initiation temperature of less than 130° C.; (1) hot tack initiation temperature of less than 130° C.; and (m) hot tack strength greater than 2 N/in.

Another alternative embodiment of the instant invention further provides a polypropylene-based film comprising: at least three layers characterized by two skin layers and at least one core layer, wherein at least one of the two skin layers consists essentially of a polymer blend of: (i) a first propylene-based polymer which comprises (A) at least 60 weight percent (wt %) units derived from propylene, and (B) from 5 to 40 wt % units derived from ethylene, and wherein the first propylene-based polymer is characterized by a Tm of less than or equal to 110° C.; and (ii) a second propylene-based polymer selected from the group consisting of rPP, hPP, and combinations thereof; and wherein the film exhibits at least one of the following characteristics: (a) change in Elmendorf tear MD of equal to or greater than 100% over a three-layer film comprising solely polypropylene consisting essentially of rPP, hPP or combinations thereof; (b) change in dart impact resistance of equal to or greater than 60% over a three-layer film comprising solely polypropylene consisting essentially of rPP, hPP or combinations thereof; (c) change in 2% secant modulus of equal to or greater than 15% over a three-layer film comprising solely polypropylene consisting essentially of rPP, hPP or combinations thereof; (d) change in puncture resistance of equal to or greater than 40% over a three-layer film comprising solely polypropylene consisting essentially of rPP, hPP or combinations thereof; (e) reduction in heat seal initiation temperature of equal to or greater than 10° C. in comparison to a three-layer film comprising solely polypropylene consisting essentially of rPP, hPP or combinations thereof; (f) reduction in hot tack initiation temperature of equal to or greater than 20° C. in comparison a three-layer film comprising solely polypropylene consisting essentially of rPP, hPP or combinations thereof; (g) normalized Elmendorf MD of equal to or greater than 25 g/mils; (h) dart impact resistance of equal to or greater than 400 g; (i) 2% Secant modulus (MD) at least 20000 psi; and (j) puncture equal to or greater than 140 ft-lb/in³; (k) heat seal initiation temperature of less than 130° C.; (1) hot tack initiation temperature of less than 130° C.; and (m) hot tack strength greater than 2 N/in.

In yet another alternative embodiment, the instant invention further provides a method for making a polypropylene-based film consisting essentially of: selecting a first propylene-based polymer; selecting a second propylene-based polymer; blending the first and second propylene-based polymers to form a polymer blend; forming the polypropylene-based film wherein at least one layer comprises the blend, wherein the first propylene-based polymer comprises from 5 to 40 wt % units derived from ethylene and the second propylene-based polymer is selected from the group consisting of rPP, hPP and combinations thereof.

### EXAMPLES

The following examples illustrate the present invention but are not intended to limit the scope of the invention. The examples of the instant invention demonstrate that the inventive films exhibit improved toughness compared to films made with 100% rPP and/or hPP while maintaining other desirable film characteristics including but not limited to clarity, strength, stiffness, sufficient elongation to break, hot tack strength, and heat seal strength.

### Inventive Examples 1-7

Inventive Examples 1-3 were each a co-extruded three layer film having the structure 10% A/80% B/10% A wherein A is a propylene-based copolymer made from 10 wt % VERSIFY3300, 88 wt % Dow PP DS6E82, and 2 wt % antiblock (AB). VERSIFY3300 is a propylene/ethylene copolymer having an MFR of 8 g/10 min, density of 0.888 g/cm³ and 5 wt % units derived from ethylene. VERSIFY 3300 is available from The Dow Chemical Company. Dow PP DS6E82, available from The Dow Chemical Company, is a random propylene/ethylene copolymer having an MFR of 8 g/10 min, density of 0.9 g/cm³ and 3.7 wt % units derived from ethylene. AB is Polybatch ABPP-10, available from A. Schulman Inc., having an MFR of 7 g/10 min, density of 0.96 g/cm³ and 0 wt % units derived from ethylene. Component B is a copolymer comprising 38 wt % VERSIFY and 62 wt % Dow PP DX5E66. Dow PP DX5E66, available from The Dow Chemical Company, is a propylene homopolymer having an MFR of 8 g/10 min and a density of 0.9 g/cm³. The grade of VERSIFY utilized in the core layer was varied in order to vary the weight % of ethylene in the core layer in Inventive Examples 1-3. Inventive Example 1 contained 9 weight % units derived from ethylene in the core layer. Inventive Example 2 contained 12 weight % units derived from ethylene in the core layer. Inventive Example 3 contained 15 weight % units derived from ethylene in the core layer.

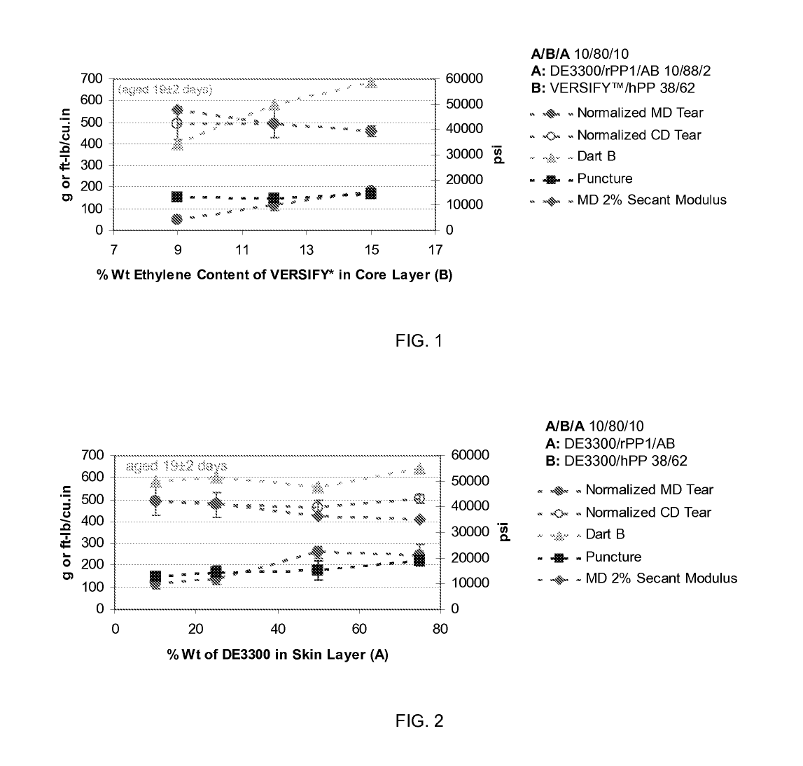

FIG. 1 illustrates the normalized Elmendorf MD and CD tear strength, dart impact resistance, Puncture and MD 2% secant modulus for each of Inventive Examples 1-3 having a film thickness of about 2 mils and before aging. In FIG. 1, the notation “DE3300” means “VERSIFY 3300.” As used herein, “before aging” means less than fifteen (15) days following formation of the film. As illustrated in FIG. 1, increasing the ethylene content used in the core layer has a significant effect on Elmendorf tear in MD, dart impact resistance and 2% secant modulus in MD. Increasing the ethylene content of the core from 9 to 15% wt provides the following improvements: (a) increases the MD Elmendorf tear of the coextruded film from 49 to 181 g/mils, an increase of over 300%; (b) increases the dart impact resistance of the coextruded film from 400 to 683 g, a 65% increase; and (c) decreases the secant modulus of the film, from 48,000 to 39,000 psi, a decrease of 25%. On the other hand, increasing the ethylene content of the core layer does not have a significant effect on the CD Elmendorf tear (5%) and puncture resistance (6%) of the coextruded film.

Inventive Examples 4-7 were each a co-extruded three layer film having the structure 10% A/80% B/10% A wherein Layer A is a propylene-based copolymer made from VERSIFY3300, Dow PP DS6E82, and antiblock (AB). Layer B is as described above in connection with Inventive Examples 1-3. The amount of the VERSIFY 3300 in Layer C is varied from 10 wt % to 75 wt %, corresponding to a total VERSIFY 3300 content in the films of from 32 wt % to 45 wt %. Inventive Example 4 contains 10 wt % units derived from VERSIFY 3300, 88 wt % units derived from Dow PP DS6E82, and 2 wt % units derived from AB in each of the skin layers. Inventive Example 5 contains 25 wt % units derived from VERSIFY 3300, 73 wt % units derived from Dow PP DS6E82, and 2 wt % units derived from AB in each of the skin layers. Inventive Example 6 contains 50 wt % units derived from VERSIFY 3300, 48 wt % units derived from Dow PP DS6E82, and 2 wt % units derived from AB in each of the skin layers. Inventive Example 7 contains 75 wt % units derived from VERSIFY 3300, 23 wt % units derived from Dow PP DS6E82, and 2 wt % units derived from AB in each of the skin layers.

FIG. 2 illustrates the normalized Elmendorf MD and CD tear strength, dart impact resistance, Puncture and MD 2% secant modulus for each of Inventive Examples 4-7 having a film thickness of about 2 mils and before aging. In FIG. 2, the notation “DE3300” means “VERSIFY 3300.” As used herein, “before aging” means less than fifteen (15) days following formation of the film. Increasing the ethylene content of each skin layer from 25 wt % to 50 wt % provides an increase the MD Elmendorf tear of the coextruded film of about 86%. Puncture resistance increased from 150 ft-lb/in³ to 220 ft-lb/in³ as the weight % of units derived from VERSIFY 3300 was increased from 10 to 75 wt %. 2% MD secant modulus decreased about 18% in increasing the VERSIFY 3300 content from 10 wt % to 75 wt %. Very little change was observed in dart impact resistance and CD Elmendorf tear over the range of VERSIFY 3300 amounts in the skin layers.

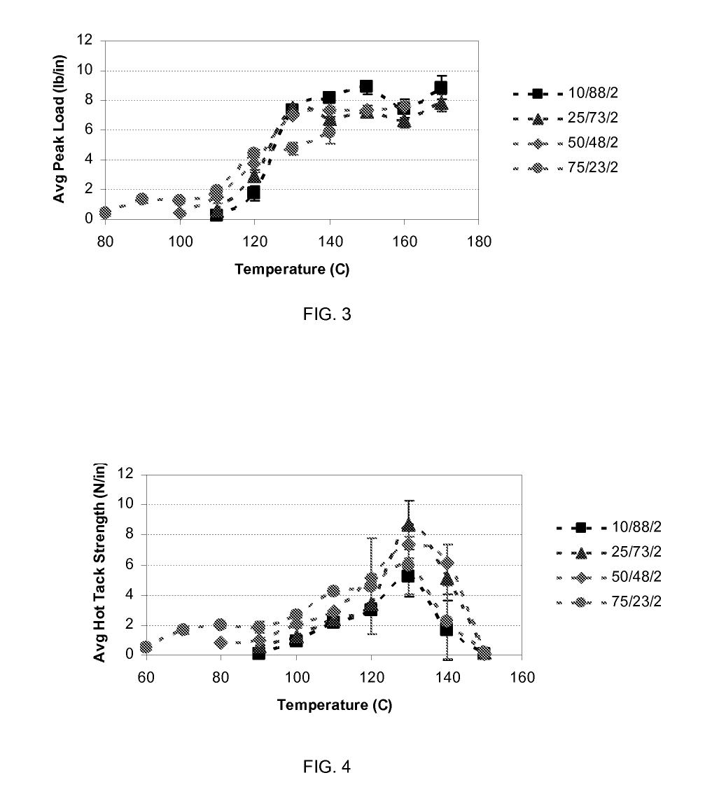

The effect of increasing VERSIFY 3300 content in the skin layers on the heat seal and hot tack properties of the coextruded films of Inventive Examples 4-7, having the same composition and film thickness as shown in FIG. 2, is shown in FIGS. 3 and 4, respectively. FIG. 3 shows that increasing VERSIFY 3300 content in the skin layers from 10 to 75% decreases the heat seal initiation temperature by approximately 10° C., from 120° C. to 110° C. at 2 lb-f/in; whereas no significant change is observed in heat seal strength, the average peak load at 160° C. is about 7 lbf/in. Similarly, FIG. 4 shows that hot tack initiation temperature decreases, approximately 20° C., with increasing VERSIFY 3300 content in the skin layer. Hot tack strength achieves an optimum value with 25% VERSIFY 3300 in each skin layer, reaching 8 N/in. Independently of the amount of VERSIFY 3300 in each skin layer, the peak hot tack strength occurs at 130° C.

Inventive Example 1-7 films are co-extruded according to the conditions shown in Table 1 below.

TABLE 1

| | Inventive |
|---|---|
| | Examples 1-7 |
| Melt Temperature (° F.) | 500 |
| Die Gap (mils) | 25 |
| Extrusion rate (lb/hr) | 440-470 |
| Line Speed (ft/min) | 300 |
| Chill roll temperature (° F.) | 70 |
| Cast roll temperature (° F.) | 70 |
| Air Knife | Yes |

### Prophetic Inventive Examples A-G, Elastic Films

A continuous coextrusion is carried out to prepare three-layer laminates with two outer layers of polypropylene and a core elastomeric layer of a styrene-isoprene-styrene block copolymer (SIS). A two inch (5.1 cm) screw diameter BERLYN extruder is used to feed the elastomer layer (KRATON 1107, available from Kraton Polymers Inc.) and a BRABENDER 1.25 inch (3.18 cm) screw diameter extruder (available from C. W. Brabender Instruments, Inc., NJ) is used to feed a blend of 70% Dow PP DX5E66 and 30% VERSIFY 3401 (having an MFR of 8 g/10 min, a density of 0.858 g/cm3 and 15 wt % units derived from ethylene), both available from The Dow Chemical Company, Midland, Mich.) into the feedblock (from CLOEREN, Inc.) and are extruded through a single manifold 18 inch (46 cm) wide film die. The film is cast onto a 60° F. (16° C.) cast roll at 14.7 ft/min (509 cm/min) at varying total caliper, as described in Table 2. Films of varying outer layer thickness are prepared.

The films are tested for relaxation by initially uniaxially stretching each sample by hand to just short of its breaking point, which is generally about 650%, releasing the sample, and observing any recovery. Recovery after initial draw is then categorized as instantaneous recovery (I), slow recovery with time (T), heat required for recovery (H) and permanent deformation (P), i.e. no significant recovery. Prophetic results are shown in Table 2 below.

The texture of the laminate is evaluated both visually and by touch after recovery and classified as fine (F), medium (M), coarse (C) or smooth (no texture discerned). The texture was also measured objectively in samples B, C and E by the periodicity (distance between folds) of the samples.

The samples are also tested for necking characteristics expressed as % change in width upon restretching of the sample. Although necking is not significant for any of these samples, generally, as skin thickness fell and the core-to-skin thickness ratio rose, necking increased.

Periodicity and C.O.F. are also shown for samples B, C and D which are both inversely related to the core/skin thickness ratio. The original C.O.F. for the samples was over 3.8, thus the microtexturing produced a significant overall reduction of C.O.F.

Though not intended to be limited by theory the ability of VERSIFY to decrease modulus in the 70% Dow PP DX5E66 and 30% VERSIFY 3401 blend compared to 100% DX5E66 is thought to be enable greater recovery of the laminate after stretching by providing less resistance to the SIS's tendency to retract. This is thought to result in enhanced elastic performance in end-use, finer texture (lower periodicity) of the folds, and lower coefficient of friction (C.O.F).

TABLE 2

| | Total | Core | Core/Skin | | |
|---|---|---|---|---|---|
| Inventive | Skin Thickness | Thickness | Thickness | | |
| Example | (μm) | (μm) | Ratio | Recovery | Texture |
| A | 5 | 90 | 18 | I | F |
| B | 8 | 145 | 18 | I | F |
| C | 12 | 175 | 14.6 | I | M-F |
| D | 7 | 54 | 7.7 | I | F |
| E | 14 | 100 | 7.1 | I | C-M |
| F | 8 | 48 | 6 | I-T | F |
| G | 20 | 45 | 2.25 | H-P | C |

### Test Methods

Test methods include the following:

MFR (Melt Flow Rate)

MFR was measured according to ASTM D1238, at 230° C. with 2.16 kg.

Elmendorf MD Tear Strength and CD Tear Strength

MD tear strength and CD tear strength were measured according to ASTM D1922. MD denotes experiment run parallel to machine direction of the film. CD denotes the experiment was run parallel to the cross direction of the film.

Clarity

Clarity was measured according to ASTM D1746.

Haze

Haze was measured according to ASTM D1003.

Gloss

Gloss was measured according to ASTM D-2457.

Heat Seal

Heat Seal was measured according to ASTM F-88.

Dart Impact Strength

Dart impact strength was measured according to ASTM D1709.

MD 2% Secant Modulus

MD 2% secant modulus was measured according to ASTM D882.

Puncture Strength

Puncture strength was measured on an INSTRON Model 4201 with SINTECH TESTWORKS SOFTWARE Version 3.10. The specimen size was “6 in×6 in,” and four measurements were made to determine an average puncture value. The film was conditioned for 40 hours after film production, and at least 24 hours in an ASTM controlled laboratory (23° C. and 50% relative humidity). A “100 lb” load cell was used with a round specimen holder of 4 inch diameter. The puncture probe is a “½ inch diameter” polished stainless steel ball (on a 2.5″ rod) with a “7.5 inch maximum travel length.” There was no gauge length, and the probe was as close as possible to, but not touching, the specimen (the probe was set by raising the probe until it touched the specimen). Then the probe was gradually lowered, until it was not touching the specimen. Then the crosshead was set at zero. Considering the maximum travel distance, the distance would be approximately 0.10 inch. The crosshead speed was 10 inches/minute. The thickness was measured in the middle of the specimen. The thickness of the film, the distance the crosshead traveled, and the peak load were used to determine the puncture by the software. The puncture probe was cleaned using a “KIM-WIPE” after each specimen.

Hot Tack Strength

Hot tack strength was measured according to ASTM F 1921, Method B. Test specimens were 1 inch in width and were conditioned as specified by ASTM E 171.

### Additional Figures

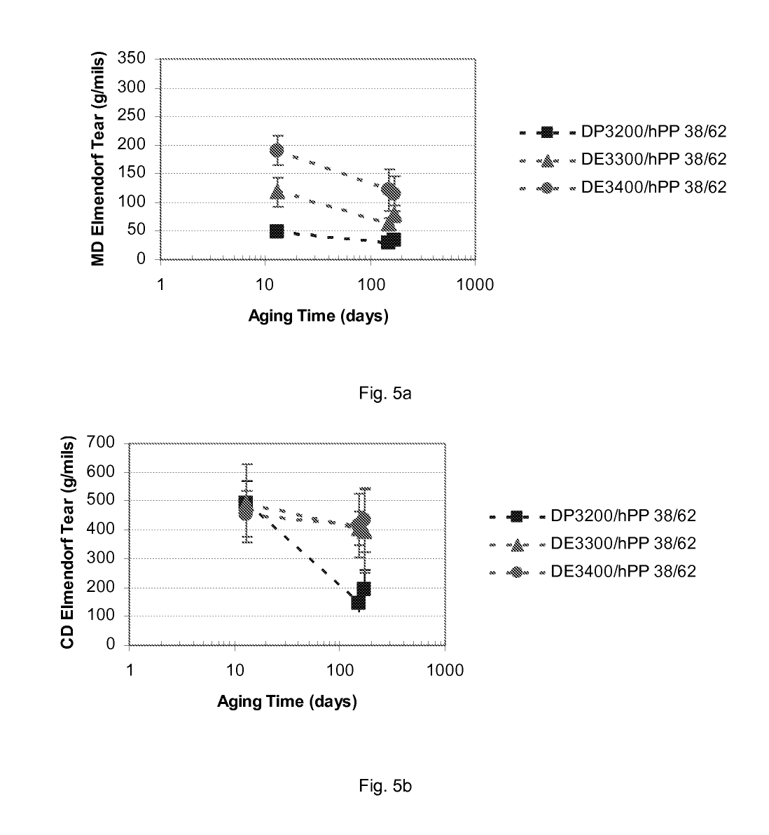

FIG. 5 shows the effect of aging on Elmendorf tear in machine and cross-directions for three layer films (A/B/A) which differ from each other only in the type of VERSIFY used in the core layer, as indicated in FIG. 5. In all cases, Elmendorf tear in both directions decreases with time. Independently of the ethylene content of the VERSIFY used in the core layer, Elmendorf tear in MD decreases approximately 40% after 170 days, FIG. 5a. This translates in higher MD tear for the coex structure with the highest ethylene content of VERSIFY, even after aging, as discussed previously in section 1.1. In the case of cross-direction, there is no initial effect on Elmendorf tear of the amount of ethylene content in VERSIFY, as discussed previously, however, with aging an effect is observed, FIG. 5b. The CD Elmendorf tear of the coex film with the lowest amount of ethylene content in the core layer, 9%, exhibits a deep decrease, approximately 70% of its initial value in 170 days. For the films with higher ethylene content values, 12 and 15%, the decrease in Elmendorf tear in CD is not as significant, in the range of 10% to 20%, in 170 days. Elmendorf tear in both directions appears to remain constant after 150 days.

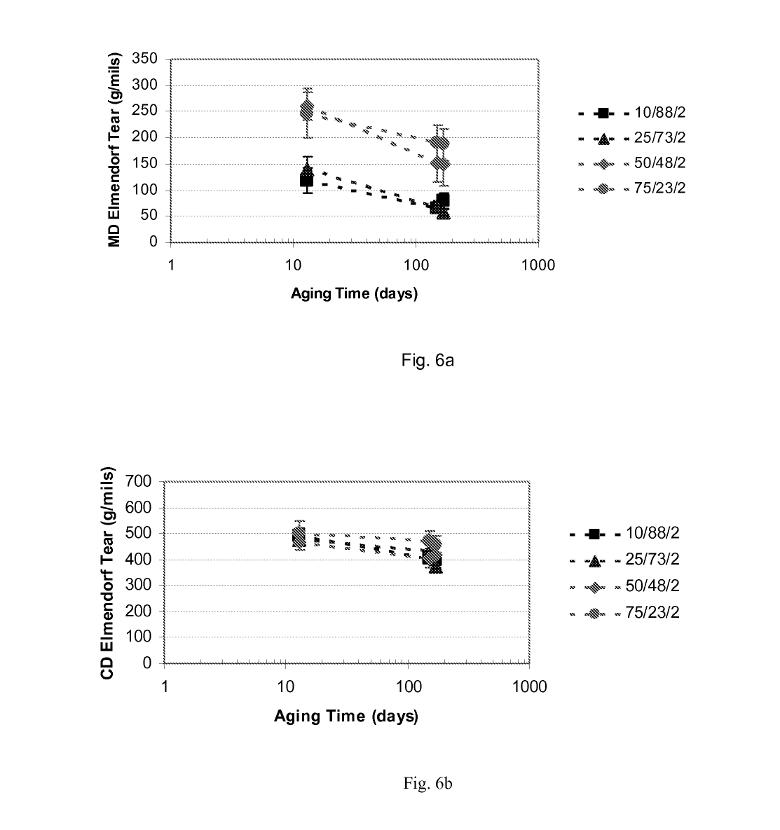

FIG. 6 shows the effect of aging on Elmendorf tear in machine and cross-directions for three layer films which differ from each other in the percentage of VERSIFY DE 3300 in the skins, increasing it from 10% to 25% to 50% to 75% of DE 3300, respectively, hence, increasing the total content of VERSIFY from 32% to 45%. As expected, in all cases, Elmendorf tear in both directions decreases with time. The change in MD Elmendorf tear with time decreases with increasing the amount of VERSIFY in the skin layer, for contents higher than 25%, FIG. 6a. The change in MD tear after aging can be reduced from 58% to 24% by increasing the VERSIFY content in the skin from 10% to 75%, therefore higher MD tear values can be obtained with higher VERSIFY content in the skin layer, as discussed previously in section 1.1. After aging, at contents below 25%, VERSIFY does not greatly modify the polypropylene as the final Elmendorf tear values are very close to those of rPP (37 g/mil) and hPP (30gr/mil). In the case of cross-direction, Elmendorf tear also decreases with time, however the change is less pronounced than that observed for tear in machine direction, FIG. 6b. As observed for MD tear, the change in CD Elmendorf tear with time, decreases with increasing the amount of VERSIFY in the skin layer. In this case, the change in CD tear after aging can be reduced from approximately 25% to 8% by increasing the VERSIFY content in the skin from 10% to 75%.

Elmendorf tear strength in cast film decreases with time, this has also been observed in blown film. The aging effect can be explained by the slow crystallization of the polypropylene; its effects are more severe on MD tear than on CD tear, due to the high orientation on machine direction that takes place during the cast film process. For the purposes of replacing a PE/PP/PE blown film, comparable values of MD Elmendorf tear after aging could only be obtained when the content of VERSIFY DE 3300 in the whole structure is at least 45%. The change in CD Elmendorf tear is less severe, however only with VERSIFY with ethylene content higher than 9% wt, the tear throughout time does not fall below the specifications for blown film.

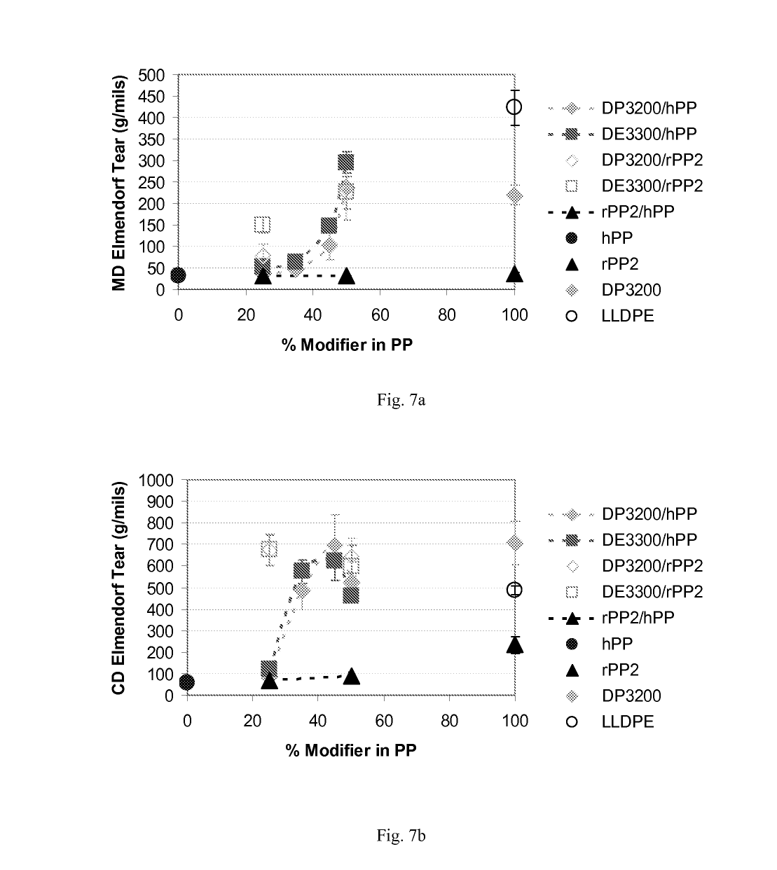

FIG. 7a describes the effect of adding VERSIFY up to 50% in hPP or rPP on normalized Elmendorf tear in machine direction. The addition of 50% VERSIFY, with either 9 or 12 wt % ethylene, increases dramatically the tear resistance of hPP. With 12 wt % ethylene content, the hPP Elmendorf tear in machine direction, increases from 30 g/mils to 300 g/mils, an increase of almost 900%, whereas with 9 wt % ethylene content, an increase of almost 700% is obtained. Following a similar trend, adding 50% VERSIFY to rPP increases its tear resistance approximately 500%. Adding more than 50% DP3200 into PP, is not likely to increase tear resistance further, since the maximum tear values obtained are very close to those of a film made entirely of DP3200. On the other hand, the addition of lower percentages of VERSIFY results more effective if rPP is used as the matrix and the higher ethylene content VERSIFY, DE3300, is used as a modifier. In this case, adding only 25% of DE3300 into rPP, results in a tear increase of already 300% as opposed to an increase of only 75% if hPP is used as the matrix. In general, VERSIFY/hPP blends exhibit higher values of tear resistance as compared to rPP/hPP blends, the difference in tear values is most significant at 50% of modifier content, tear values with VERSIFY are almost 10 times higher than those obtained with rPP, probably a result of the lower ethylene content in commercial random polypropylene, 5.7%. The maximum values of Elmendorf tear obtained by adding VERSIFY into either hPP or rPP are lower, about 30%, than those obtained with 100% LLDPE.

Similarly, adding VERSIFY into hPP or rPP also increases cross-direction tear, as observed in FIG. 7b. In this case, with the addition of 45% VERSIFY, hPP tear increases from 60 g/mils to a maximum of about 660 g/mils. For rPP, the maximum tear resistance is obtained with only 25% VERSIFY, an increase from 230 g/mils to about 700 g/mils. Therefore to obtain a high tear resistance in cross-direction, the addition of VERSIFY to rPP is more effective than its addition to hPP. In both cases, the maximum cross-direction tear values are very close to that of a monolith film of DP3200. As observed for machine direction tear, VERSIFY increases hPP tear more effectively than rPP; probably due to the lower ethylene content in commercial rPP. For cross-direction tear, the maximum values obtained by adding VERSIFY into either hPP or rPP are 40% higher than those obtained with 100% LLDPE.

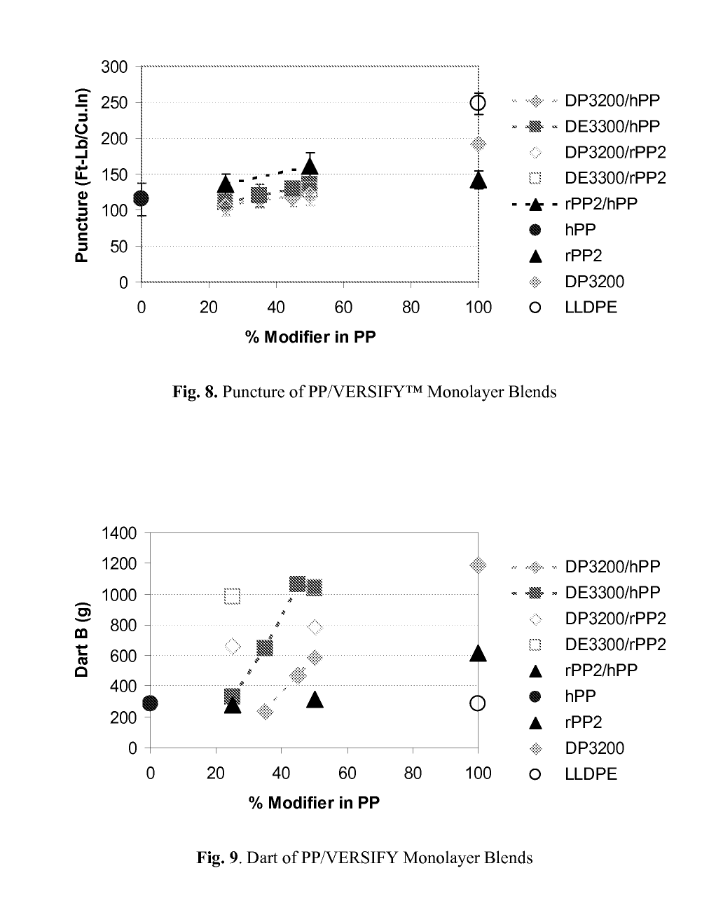

The effect of adding VERSIFY in the puncture resistance of the polypropylene cast films is summarized in FIG. 8. Adding up to 50% VERSIFY into hPP results in a slight increase in puncture resistance, about 20%, from 120 to 140 ft-lb/in³.

FIG. 9 shows that increasing VERSIFY content in hPP or rPP increases dart resistance significantly; a higher improvement is observed with increasing ethylene content in VERSIFY. Adding 50% of VERSIFY with 9% ethylene content increases 100% the dart resistance of a hPP film, whereas adding the same amount of VERSIFY with 12% ethylene content increases the dart resistance of hPP to approximately 260%.

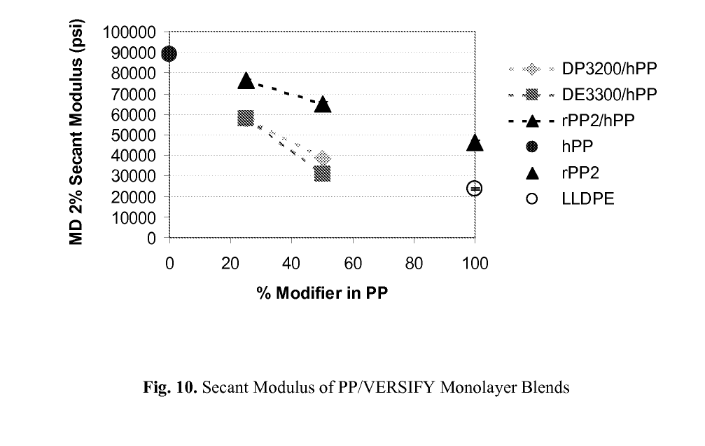

The addition of VERSIFY into hPP decreases linearly the 2% secant modulus of hPP in machine direction, FIG. 10. Adding 50% DP 3300 into hPP decreases its 2% secant modulus by 65%, from 89,000 to 31,000 psi. Adding 50% rPP into hPP decreases its modulus by only 27%. The modulus of a 50% VERSIFY/hPP blend is at least 30% higher than that of LLDPE.

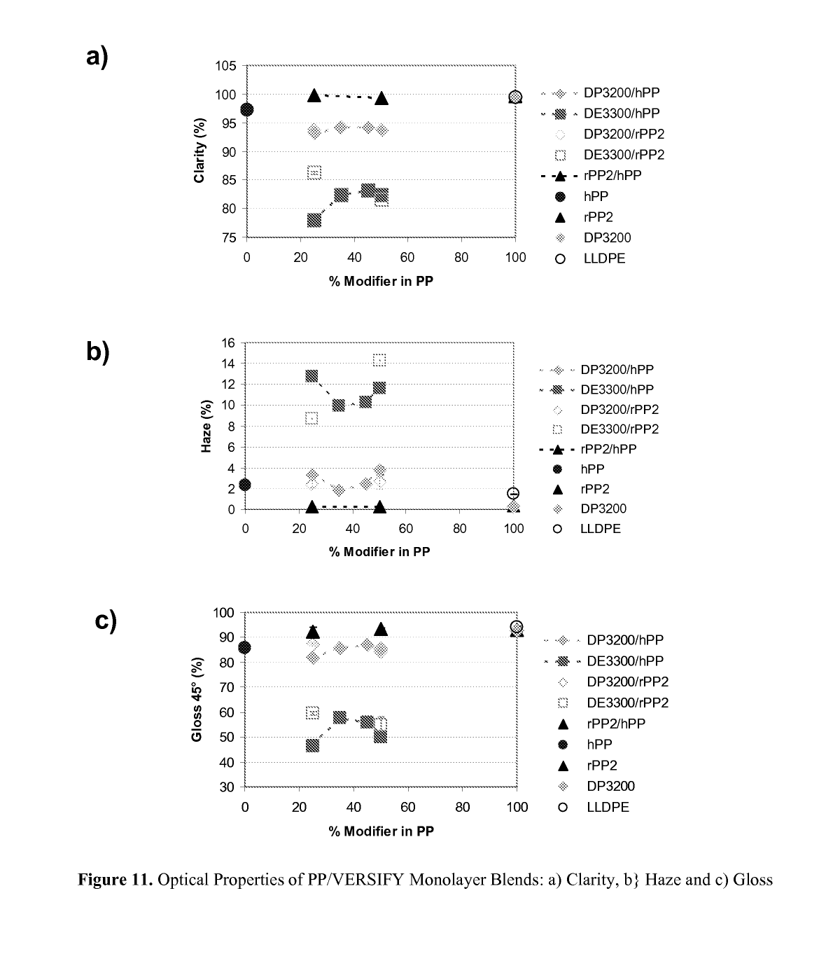

FIG. 11 shows the optical properties of PP/VERSIFY monolayer cast films. The addition of VERSIFY with 9% ethylene content into PP does not alter significantly the optical properties of hPP or rPP. In the case of hPP, clarity and gloss decrease slightly, approximately 3 and 1% respectively; haze increases about 1%. On the other hand, the addition of VERSIFY with higher ethylene content, 12 wt %, results detrimental to the optical properties of PP. For hPP blends, the addition of 35% VERSIFY 3300 decreases significantly the optical properties of the homopolymer; clarity and gloss decrease approximately 14 and 27% respectively; haze increases about 10%; increasing further the amount of VERSIFY to 50% results in slightly lower optical properties than with 35%. A similar trend is observed for blends of DP 3300 and random polypropylene; however the best optics of the DP3300 blends are obtained by adding only 25% into rPP. In contrast, the optical properties of hPP are maintained with the addition of rPP even to 50%, due to the lower ethylene content in rPP. LLDPE has comparable optical properties to rPP.

In summary, the optical properties of PP/VERSIFY blends are optimized by using VERSIFY with 9% wt ethylene content; addition of a 12% wt ethylene content VERSIFY results detrimental to he optical properties of PP. Similar optical properties to LLDPE are obtained with PP/VERSIFY DP 3200.

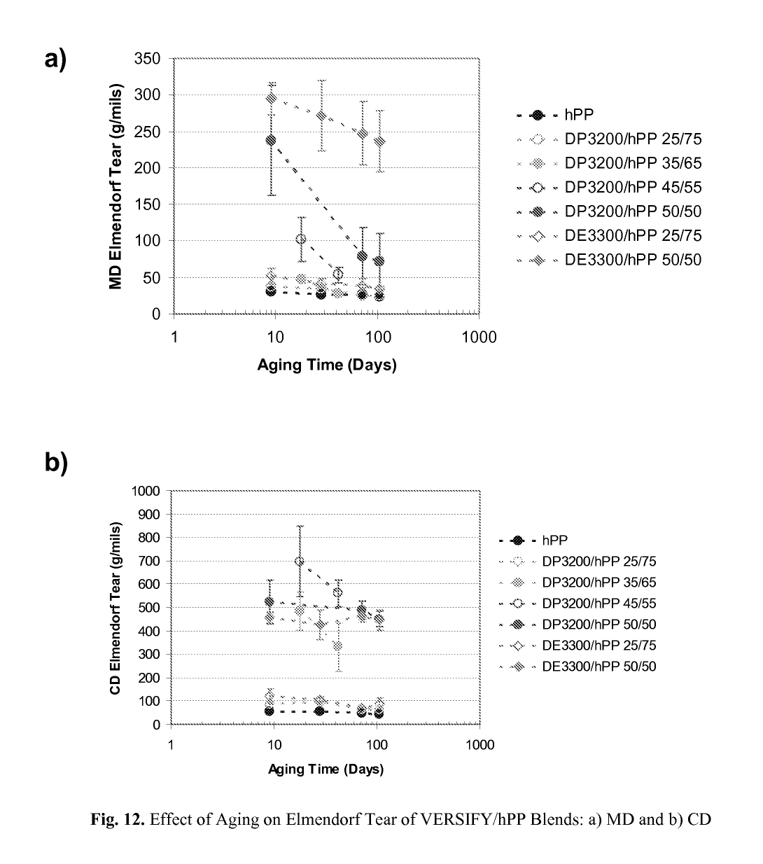

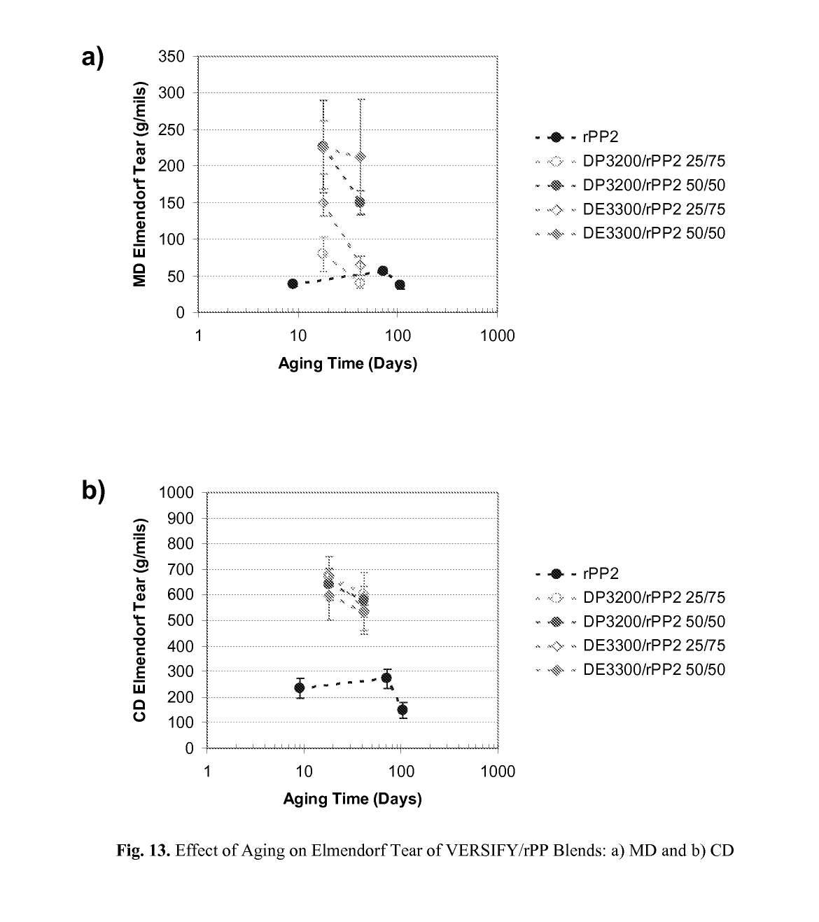

FIGS. 12 and 13 show the effect of aging on Elmendorf tear of VERSIFY/hPP blends and VERSIFY/rPP blends, respectively. As observed in the case of coex films, Elmendorf tear decreases with time for all blends, the change is more pronounced in machine direction than in cross direction.

VERSIFY/hPP blends up to 35% VERSIFY content exhibit MD tear values through time less than 35 gr/mil, FIG. 12a. Even a blend with 45% VERSIFYcontent, exhibits a drastic decrease in MD tear reaching 50 gr/mil after 42 days. 50/50 VERSIFY/hPP blends exhibit initial MD tear values greater than 200 gr/mil, however even with 50% VERSIFYDP 3200, a 70% change in MD tear throughout 70 days is observed. Only with 50% of the higher ethylene content VERSIFYDE 3300 (12% wt. ethylene), MD tear is maintained relatively constant throughout time, showing only a change of 20% after 100 days, keeping the MD tear value above 200 gr/mil. Similarly, hPP blends with less than 50% VERSIFYcontent, exhibit a more significant decrease in tear in cross-direction than those with 50%, FIG. 12b. Throughout time, blends with 25% VERSIFYdoes not exhibit any significant improvement in Elmendorf tear compared to 100% homopolymer, regardless of the VERSIFYgrade; all values are below 100 gr/mil. Blends with 35% and 45% VERSIFYDP3200 in hPP exhibit higher initial tear values but in both cases, the drop with time is significant, 20% and 32% respectively after 24 days. The 50/50 VERSIFYrPP blends exhibit the least change in CD tear values with time, being the blend with VERSIFYDE 3300 constant around 450 gr/mil.

Similar to the blends of VERSIFY with homopolymer polypropylene, blends with random polypropylene follow the same trend in Elmendorf tear with time, FIG. 13. In machine direction, random polypropylene does not offer a significant advantage over homopolymer, tear values are very similar. In cross-direction, the change in tear after 24 days between blends with 25 and 50% VERSIFY is not markedly different as in all other cases previously discussed. For the 50/50 blends the reduction is in the 10% range while in the 25/75 blends the reduction is in the 20% range.

The present invention may be embodied in other forms without departing from the spirit and the essential attributes thereof, and, accordingly, reference should be made to the appended claims, rather than to the foregoing specification, as indicating the scope of the invention.

1. A polypropylene-based film comprising:
at least three layers characterized by two skin layers and at least one core layer,
wherein the at least one core layer comprises a polymer blend of:
(i) a first propylene-based polymer which comprises (A) at least 60 weight percent (wt %) units derived from propylene, and (B) from 5 to 20 wt % units derived from ethylene, and wherein the first propylene-based polymer is characterized by a melting temperature (Tm) of less than or equal to 110° C., and
(ii) a second propylene-based polymer selected from the group consisting of rPP and hPP, and
wherein the film exhibits at least one of the following characteristics:
(a) change in Elmendorf tear MD of equal to or greater than 100% over a three-layer film comprising solely polypropylene consisting essentially of rPP, hPP or combinations thereof;
(b) change in dart impact resistance of equal to or greater than 60% over a three-layer film comprising solely polypropylene consisting essentially of rPP, hPP or combinations thereof;
(c) change in 2% secant modulus of equal to or greater than 15% over a three-layer film comprising solely polypropylene consisting essentially of rPP, hPP or combinations thereof; and
(d) change in puncture resistance of equal to or greater than 40% over a three-layer film comprising solely polypropylene consisting essentially of rPP, hPP or combinations thereof.

2. A polypropylene-based film comprising:
at least three layers characterized by two skin layers and at least one core layer,
wherein at least one of the two skin layers comprises a polymer blend of:
(i) a first propylene-based polymer which comprises (A) at least 60 weight percent (wt %) units derived from propylene, and (B) from 5 to 40 wt % units derived from ethylene, and wherein the first propylene-based polymer is characterized by a Tm of less than or equal to 110° C.; and
(ii) a second propylene-based polymer; and
wherein the film exhibits at least one of the following characteristics:
(a) change in Elmendorf tear MD of equal to or greater than 100% over a three-layer film comprising solely polypropylene consisting essentially of rPP, hPP or combinations thereof;
(b) change in dart impact resistance of equal to or greater than 60% over a three-layer film comprising solely polypropylene consisting essentially of rPP, hPP or combinations thereof;
(c) change in 2% secant modulus of equal to or greater than 15% over a three-layer film comprising solely polypropylene consisting essentially of rPP, hPP or combinations thereof;
(d) change in puncture resistance of equal to or greater than 40% over a three-layer film comprising solely polypropylene consisting essentially of rPP, hPP or combinations thereof;
(e) reduction in heat seal initiation temperature of equal to or greater than 10° C. in comparison a three-layer film comprising solely polypropylene consisting essentially of rPP, hPP or combinations thereof; and
(f) reduction in hot tack initiation temperature of equal to or greater than 20° C. in comparison a three-layer film comprising solely polypropylene consisting essentially of rPP, hPP or combinations thereof.

3. The polypropylene-based film according to claim 1, wherein the film is an extrusion coating and the first polypropylene-based polymer has an MFR of from 8 to 40 g/10 min.

4. The polypropylene-based film according to claim 1, wherein the film is a blown film and the first polypropylene-based polymer has an MFR of from 0.3 to 10 g/10 min.

5. The polypropylene-based film according to claim 1, wherein the film comprises three layers characterized by two skin layers and one core layer.

6. The polypropylene-based film according to claim 1, wherein the film comprises four layers characterized by two skin layers and two core layers.

7. The polypropylene-based film according to claim 1, wherein the film comprises five layers characterized by two skin layers and three core layers.

8. The polypropylene-based film according to claim 1, further comprising at least one tie layer disposed between at least one skin layer and at least one core layer.

9. The polypropylene-based film according to claim 1, wherein the total skin thickness comprises from 4 to 40 percent of the total thickness of the film.

10. A method of making the polypropylene-based film according to claim 1, comprising:
selecting a first propylene-based polymer;
selecting a second propylene-based polymer;
blending the first and second propylene-based polymers to form a polymer blend;
forming the polypropylene-based film wherein at least one layer comprises the blend,
wherein the first propylene-based polymer comprises from 5 to 40 wt % units derived from ethylene and the second propylene-based polymer is selected from the group consisting of rPP, hPP and combinations thereof.

11. The method according to claim 10 wherein the film is formed by a co-extrusion method selected from the group consisting of cast extrusion, extrusion blowing and extrusion coating.

12. The method according to claim 10 wherein the film is formed by a lamination method selected from the group consisting of thermal, ultrasonic and adhesive lamination.

13. An article comprising the propylene-based film according to claim 1.

14. The article according to claim 13, wherein the article is selected from the group consisting of food packaging, household packaging, coated fabrics, elastic films and fibers.

15. The article according to claim 13, wherein the article is a hygiene film.
